(19) United States
(12) Patent Application Publication
(10) Pub. No.: US 2019/0059311 A1
(43) Pub. Date: Feb. 28, 2019

(54) TOLERANT IMPATIENS VARIETIES AND METHOD OF BREEDING THE SAME

(71) Applicant: Kerley & Co., Cambridge (GB)

(72) Inventors: David KERLEY, Cambridge (GB); Priscilla KERLEY, Cambridge (GB); Sarah KERLEY, Cambridge (GB); Tim KERLEY, Cambridge (GB)

(21) Appl. No.: 15/959,616

(22) Filed: Apr. 23, 2018

Related U.S. Application Data
(60) Provisional application No. 62/488,271, filed on Apr. 21, 2017.

(51) Int. Cl. A01H 6/16; C12N 15/82

(52) U.S. Cl. CPC A01H 6/165; C12N 15/82

(57) ABSTRACT

Provided herein are Impatiens walleriana x gordonii cultivars which exhibit tolerance to Plasmopara obducens, the cause of Impatiens downy mildew. Methods for breeding the disclosed Impatiens are also provided.

This application claims the benefit of U.S. Provisional Patent Application No. 62/488,271, filed Apr. 21, 2017, the entirety of which is incorporated herein by reference.

### BACKGROUND OF THE INVENTION

### 1. Field of the Invention

The present embodiments provided herein relate generally to horticultural, plant breeding and plant genetics.

### 2. Description of Related Art

Impatiens walleriana has historically been one of the most important bedding plant crops in the U.S.A. and European Union. However, since the outbreak of the disease Impatiens Downy Mildew (IDM), caused by Plasmopara obducens, sales of Impatiens have dropped significantly. The disease causes defoliation and plant collapse. There has been no known resistance or tolerance to the disease in Impatiens walleriana.

IDM was found affecting Impatiens walleriana for the first time in the United Kingdom in 2003 and remained absent or at low levels until significant levels of infection were again reported in 2007, 2008 and 2011. Initially listed as a notifiable disease, a decision was taken by Defra Plant Health to remove the need for emergency control measures and allow the industry to manage the disease in a similar manner to other downy mildew diseases on ornamentals. AHDB Horticulture funded work provided an understanding of the disease and its control showing that fungicides could provide effective protectant control; with fungicides containing metalaxyl-M providing the most effective control. However, in 2011 the widespread introduction of a strain of P. obducens resistant to metalaxyl-M into the U.K. meant that control of the disease through use of fungicide became more problematic.

Regional outbreaks of IDM were seen for the first time in North America in summer 2011. In early January 2012, outbreaks of impatiens downy mildew were observed in landscape beds and greenhouses in south Florida. It was initially unclear whether this was a continuation of the 2011 outbreaks or a new cycle of disease for 2012. By the end of the 2012 season, IDM had been confirmed in 34 states.

### SUMMARY OF THE INVENTION

In a first embodiment there is provided an Impatiens hybrid plant which exhibits tolerance to Plasmopara obducens infection, wherein the plant is obtained by introgression of the tolerance trait from a plant grown from the seed deposited at NCIMB under the accession no. 42708, said plant having been selected for a Plasmopara obducens tolerant trait. In some aspects, the plant exhibits infection less than 80% of the time after inoculation with Plasmopara obducens. In further aspects, the plant exhibits infection less than 70%, 60%, 50%, 40%, 30%, 20%, 10% or 5% of the time after inoculation with Plasmopara obducens. In certain aspects, the plant exhibits complete tolerance to Plasmopara obducens infection. Thus, in some aspects, a plant of the embodiments is at least about 20%, 30%, 40%, 50%, 60%, 70%, 80%, 90% or 95% resistant to signs of infection after inoculation with Plasmopara obducens.

A further embodiment provides a progeny plant of an Impatiens hybrid plant in accordance with embodiment and aspects described above, wherein said progeny plant exhibits tolerance to Plasmopara obducens infection.

In still a further embodiment, there is provided a tissue culture of regenerable cells of the Impatiens hybrid plant of any of the embodiments and aspects described herein. In several aspects, the regenerable cells are from embryos, meristematic cells, pollen, leaves, petals, roots, root tips, anther, pistil, seed or stem.

Still yet a further embodiment provides a plant part of the Impatiens hybrid plant of any of the embodiments and aspects described herein. In certain aspects, the part is a seed, a stalk, a petal, a bud, a leaf or a root. In some aspects, said part can be regenerated into a plant which exhibits tolerance to Plasmopara obducens infection.

In yet a further embodiment, there is provided a method of producing an Impatiens hybrid plant comprising crossing a plant of the embodiments and aspects described above with a second Impatiens plant and selecting a progeny plant which exhibits tolerance to Plasmopara obducens infection.

Another embodiment provides a method for obtaining an Impatiens hybrid plant according to the embodiments and aspects described above comprising the step of introgression of the tolerance trait from a plant grown from the seed deposited at NCIMB under the accession no. 42708.

In still yet a further embodiment, there is provided an Impatiens seed wherein, upon growth, the seed produces a plant which exhibits a tolerance to Plasmopara obducens infection, said tolerance trait having been introgressed from a plant grown from the seed deposited at NCIMB under the accession no. 42708.

As used herein in the specification and claims, “a” or “an” may mean one or more. As used herein in the specification and claims, when used in conjunction with the word “comprising”, the words “a” or “an” may mean one or more than one. As used herein, in the specification and claims, “another” or “a further” may mean at least a second or more.

Other objects, features and advantages of the present invention will become apparent from the following detailed description. It should be understood, however, that the detailed description and the specific examples, while indicating certain embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-C. Examples of sporulation on underside of leaves on line 15-178-1 following the assessment at time point 1 (1A) and at time point 2, 6 days later (1B and 1C). Circled areas highlight sporulation on the underside of leaves.

### DESCRIPTION OF ILLUSTRATIVE EMBODIMENTS

### I. The Present Embodiments

‘Ray of Hope’ (RoH) is a cross between Impatiens gordonii and I. walleriana. The production of this hybrid plant does not require collection of material from the wild (Griffiths and Matatiken, 2013). The date of hybridisation is unknown but it was available as reported by the Guardian newspaper in May 2004 (theguardian.com/lifeandstyle/2004/may/02/ethicalliving.gardens). As described herein, this species hybrid was initially identified to exhibit minimal tolerance to IDM. By means of cross pollination over several years, the tolerance has been strengthened to such a degree that plants in the landscape look healthy for the whole season whilst nearby plants collapse completely with IDM. One variety tested under controlled conditions even showed complete tolerance to the disease.

### II. DEFINITIONS

As used herein, “susceptibility” or “susceptible” refers to inability of a plant variety to restrict the growth and/or development of a specified pest.

As used herein, “resistance” refers to the ability of a plant variety to restrict the growth and/or development of a specified pest and/or the damage it causes when compared to susceptible plant varieties under similar environmental conditions and pest pressure.

Resistant varieties may exhibit some disease symptoms or damage under heavy pest pressure. Two levels of resistance are defined. As used herein “high resistance” (HR) refers to plant varieties that highly restrict the growth and/or development of the specified pest and/or the damage it causes under normal pest pressure when compared to susceptible varieties. These plant varieties may, however, exhibit some symptoms or damage under heavy pest pressure. As used herein “intermediate resistance” (IR) refers to plant varieties that restrict the growth and/or development of the specified pest and/or the damage it causes but may exhibit a greater range of symptoms or damage compared to high resistant varieties. Intermediate resistant plant varieties will still show less severe symptoms or damage than susceptible plant varieties when grown under similar environmental conditions and/or pest pressure. Varieties claiming the same level of resistance against a specific pest may exhibit a different resistance response due to a different genetic makeup of a variety.

As used herein, “immunity” refers to when a plant is not subject to attack or infection by a specified pest.

As used herein, “single” or “single-type” are each defined as the typical Impatiens plant which produces flowers having five petals per flower or the typical Impatiens flower which has five petals.

As used herein, “semi-double” or “semi-double-type”, are each defined as a Impatiens plant which produces one or more flowers having a sixth full or partial petal per flower or a Impatiens flower which has a sixth full or partial petal.

As used herein, “double”, “double-type” or “double-flowering” are each defined as a Impatiens plant which produces one or more flowers having at least 7 full or partial petals per flower or a Impatiens flower which has at least 7 full or partial petals. Double-flowering Impatiens cultivars are genetically stable. Double-flowering cultivars can be stably reproduced by means of asexual propagation. The characteristic of doubleness can be predictably bred into diverse single-type and semi-double-type Impatiens genetic backgrounds.

Horticultural elite—Plants exhibiting desired horticultural traits are considered to be horticultural elite, viz. genetic traits. Traits that may be considered to confer elitism are good longevity, large blooms and/or tolerance to pests, tolerance to disease, long flowering time, and the like.

Genetic transformation—A process of introducing a DNA molecules (e.g., a vector or expression cassette) into a cell or protoplast in which that exogenous DNA is incorporated into a chromosome or is capable of autonomous replication.

Introgression—The process of transferring a genetic trait (e.g., a resistance phenotype) from one genotype to another.

### III. DEPOSIT INFORMATION

A representative deposit of 1,100 seeds from Impatiens walleriana x gordonii hybrid plants from hybrid population 16-Z2 has been made with the National Collections of Industrial, Food and Marine Bacteria (NCIMB), Ferguson Building, Craibstone Estate, Bucksburn, Aberdeen AB2 9YA, Scotland, United Kingdom on Jan. 5, 2017. Those deposited seeds have been assigned Accession No. NCIMB 42708.

The foregoing deposit was made in accordance with the terms and provisions of the Budapest Treaty relating to deposit of microorganisms and were made for a term of at least thirty (30) years and at least five (05) years after the most recent request for the furnishing of a sample of the deposits is received by the depository, or for the effective term of the patent, whichever is longer, and will be replaced if it becomes non-viable during that period.

### IV. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Breeding of IDM Tolerant Impatiens walleriana x gordonii Hybrid Plants

‘Ray of Hope’ (‘RoH’, Impatiens walleriana x I. gordonii) plants were obtained (Garden Bargains, ordered on Dec. 5, 2011). RoH plants were planted in summer 2011 in outdoor containers at Kerley & Co., alongside Kerley & Co. bred I. walleriana and other commercial varieties from other breeding companies (both I. walleriana and I. walleriana hybrids). The Kerley & Co. trial site became infected with IDM. Although IDM had been previously known in the U.K., it had never before been seen on site at Kerley & Co. Observing the trial outdoor containers, it was noticed that the RoH plants were the last variety to show symptoms of the disease and generally looked healthier for longer than all the other varieties in the trial.

In late summer 2011, RoH was crossed with six different selections of I. walleriana in a controlled breeding program with the intention of breeding plants with enhanced tolerance to IDM. Hand pollinations were made under glass using RoH as the female parent. The seeds from each cross were harvested, cleaned and stored separately.

In spring 2012, seeds from the crosses were sown into seed pots. After germination, seedlings were pricked out into 6-packs and grown on to flowering stage in a greenhouse environment. No fungicides were applied to allow natural infection of IDM. This did occur and symptoms of IDM infection were observed. At this point, plants were selected for apparent tolerance to IDM by choosing those plants with little or no sporulation on the undersides of the leaves. Within the populations, some plants were found with a degree of tolerance to IDM. The percentage of tolerant plants ranged from 0% to 17% tolerance within a population. No further testing was done on the plants in 2012. A fungicide rotation was applied so plants could be kept for a trial the following year. Additionally, the selected plants showing tolerance were backcrossed to RoH over the summer.

In spring 2013, the plants selected in 2012 were multiplied by means of vegetative cuttings. Plants were planted out and established in open ground during May. No fungicides were applied and the plants were assessed visually throughout the season for visual symptoms of IDM.

Seedlings of the backcrosses were grown and selected in the same way as in 2012. After selection a cutting was taken from each plant to maintain a clean plant in the greenhouse, then the seedling was planted in the open ground with the 2012 selections and treated and assessed in the same way. At the initial seedling selection, the percentage of plants showing a degree of tolerance was between 0% and 51% but normally around 20%. During subsequent evaluations only the most tolerant seedling selections were kept.

In summer 2013, a few crosses were made, mostly out crossing to material with no tolerance. All seedlings proved to be highly susceptible and were subsequently discarded. One cross was made between two “unrelated” individuals showing good tolerance to IDM (“unrelated” lines all share RoH as a common parent in the pedigree but no other shared parent). For this cross, 26% of the seedlings showed a degree of tolerance.

In summer 2014, vegetatively propagated plants of selections from previous years were planted again in the open field and visually assessed. The most tolerant were kept, others less so were discarded. The following types of crosses were made:
Backcross tolerant varieties to RoH
F2 of F1 RoH crosses made in 2012 and 2013
‘Unrelated’ tolerant lines X.
Outcross of tolerant varieties to varieties with no tolerance (to introduce new colours).

In spring 2015, seedlings were grown and selected as in previous years. However, once selections had been made they were not planted in the open field but potted into 1L pots and kept in the greenhouse. No fungicides were applied and IDM infection was observed throughout the season. Less tolerant selections were discarded. In the initial selection, between 0 and 18% of seedlings demonstrated some level of tolerance. In terms of giving a higher % tolerance within a population, generally the cross types were ranked as follows:
1. Unrelated lines X
2. Backcross to RoH
3. F2 of Fl RoH crosses made in 2012 and 2013
4. Outcross to varieties with no tolerance (although 12-71-1 is an F1 cross between RoH and a variety with no tolerance, but showed complete resistance)

The results suggested the tolerance was not a single gene resistance but most likely a multi-gene field resistance. Further crosses were made, crossing unrelated lines, backcrossing to RoH and making a F2 generation.

In December 2015, liners of two varieties (12-71-1 and 15-178-1) known to have a high degree of tolerance were sent to FERA for controlled testing for tolerance to the metylaxyl-resistant strain of IDM as described below in Example 2.

In spring 2016, seedlings were grown and selected as in 2015. All selections remaining from previous years (2012-2015) were vegetatively reproduced and four young plants planted in the same piece of open ground as in 2015 (known to be carrying the IDM spores) and visually assessed throughout the season.

193 different varieties were grown in plots. By early October, four varieties showed no sporulation or symptoms of IDM. 37 varieties showed slight or very slight sporulation on the undersides of the leaves but visually looked healthy. RoH was also planted.

Generally, the selections made in later years showed more tolerance than earlier selections and were more vigorous. 12-71-1 was, however, an exception. Many selections were more tolerant than RoH. RoH had sporulation evident by the end of July. At the same time, 111 out of 192 (58%) Kerley & Co. selections were showing no IDM symptoms.

Between 2012 and 2015 an estimated 7200 seedlings have been grown and assessed for IDM tolerance. Of those, 4 varieties appear to have very high tolerance (possibly complete resistance) to IDM. This includes breeder code 12-71-1 which was tested at FERA (see Example 2). 37 varieties have high tolerance to IDM as evidenced by visual healthy appearance and only slight or very slight sporulation on the undersides of leaves at the end of the season.

### Example 2

### Evaluation of Plants for Tolerance to P. obducens

Evaluation of several hybrid plant lines was conducted by Fera under contract.

Material and Methods—Twelve impatiens liner plants of line 12-71-1 and eleven of 15-178-1 arrived at Fera on the Sep. 12, 2015 and were immediately potted into 9 cm pots containing a peat based compost in 5% loam from Petersfield. The plants were generally in good condition although some leaves on most plants showed signs of damaged.

The liner plants were inoculated (Dec. 9, 2015, after potting on) using a sporangial suspension of Plasmopara obducens isolate 18 (a metalaxyl-M resistant isolate collected in 2011). The suspension was produced by washing sporangia from the leaves of a previously infected plant, counting the number of sporangia using a counting chamber and adjusting the sporangial concentration to approximately 10⁴ sporangia ml^{-1.}

Six plants from lines 12-71-1 and 15-178-1 and six of Dezire White (a control variety known to be sensitive to infection by P. obducens) were inoculated to run-off with the sporangial suspension. The remaining plants from the test lines (six of 12-71-1 and five of 15-178-1) and six of Dezire White were sprayed with an equivalent volume of sterile distilled water to provide uninoculated controls. All inoculated and control plants were incubated for approximately 18 hrs in the dark at 5° C.; incubation was in propagator tops to prevent drying out of inoculum. Plants were then transferred to a glasshouse and grown at 20° C. for 8 days. During this period plants were watered from below to ensure sporangia were not produced prematurely. Sporangial production was then initiated (Dec. 17, 2015) by wetting the upper surface of leaves and incubating overnight in a propagator top. All plants were assessed (Dec. 18, 2015) for the production of sporangia and the percentage leaves infected calculated.

Following the first assessment the inoculated plants were split into two sets of three, with the first set left to see whether symptom expression took longer in the test plants than the Dezire White control and the second set re-inoculated (Dec. 18, 2015). The first set of plants was re-assessed five days after the initial assessment (Dec. 24, 2015) and the re-inoculated plants 12 and 18 days after the second inoculation (Dec. 30, 2015) and May 1, 2016 respectively).

Results—Plants from impatiens lines 12-71-1, 15-178-1 and the controls were first assessed for downy mildew symptoms nine days after inoculation. All six control plants showed signs of sporulation, with the percentage leaves affected ranging from 20% to 95% (Table 1). None of the six plants inoculated from test line 12-71-1 showed any signs of sporulation (Table 1). One plant from line 15-178-1 died in the period between inoculation and the first assessment, of the remaining five plants inoculated one was infected with sporulation observed on 40% of leaves (Table 1).

TABLE 1
Infection of plants from test lines 12-71-1 and 15-178-1 following inoculation with a
metalaxyl-M resistant strain of Plasmopara obducens.

| | | | Control* | | | | | | 12-7-1 | | | | | | 15-178-1 | | | | | |
|---|---|---|---|---|---|---|---|---|---|---|---|---|---|---|---|---|---|---|---|---|
| Assessment | | | 1 | 2 | 3 | 4 | 5 | 6 | 1 | 2 | 3 | 4 | 5 | 6 | 1 | 2 | 3 | 4 | 5 | 6 |
| 1st | 1st assessment | Plant infected | ✓ | ✓ | ✓ | ✓ | ✓ | ✓ | x | x | x | x | x | x | x | x | x | ✓ | x | Died |
| inoculation | (10/12/15) | Sporulating leaves (%) | 95 | 20 | 45 | 50 | 40 | 20 | 0 | 0 | 0 | 0 | 0 | 0 | 0 | 0 | 0 | 40 | 0 | — |

| | | | | | | | |
|---|---|---|---|---|---|---|---|
| 2nd assessment | Plant infected | x | X | x | x | ✓ | x |
| (24/12/15) | Sporulating leaves (%) | 0 | 0 | 0 | 0 | 100 | 0 |

| | | | | | | | | | | | |
|---|---|---|---|---|---|---|---|---|---|---|---|
| 2nd | 1st assessment | Plant infected | ✓ | x | x | x | x | x | ✓ | ✓ | — |
| inoculation | (30/12/15) | Sporulating leaves (%) | 47 | 0 | 0 | 0 | 0 | 0 | 100 | 73 | — |
| | 2nd assessment | Plant infected | ✓ | ✓ | ✓ | x | x | x | — | ✓ | — |
| | (05/01/16) | Sporulating leaves (%) | 60 | 10 | 30 | 0 | 0 | 0 | — | 80 | — |

*a fresh set of three control plants were inoculated when the test lines were inoculated for a second time.
✓ = infected plant.
x = uninfected plant.

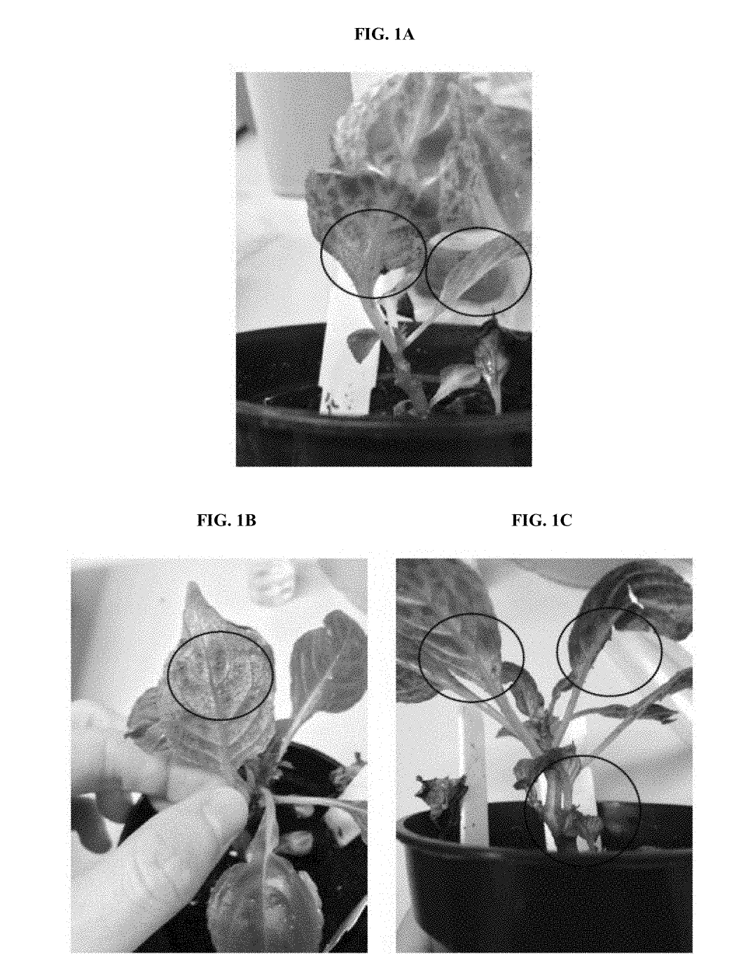

To determine whether infection in the test lines took longer to express than in the control, three of the inoculated plants (replicate plants 1 to 3) from each test line were re-assessed for sporulation six days after the initial assessment. As in the first assessment no plants from line 12-71-1 showed any signs of sporulation (Table 1) however one of the three plants from line 15-178-1 was infected with sporulation observed on all leaves (Table 1; FIG. 1A).

The plants not re-assessed were re-inoculated immediately after the first assessment; at the same time a second set of three control plants were also inoculated. The re-inoculated plants and controls were then assessed for sporulation 12 and 18 days later. By the second assessment (May 1, 2016) sporulation was observed on all three control plants with sporulation occurring on between 10 and 30% of leaves. No sporulation was observed on any of the re-inoculated plants in test line 12-71-1 on either of the assessments dates. Sporulation was observed on both re-inoculated plants of test line 15-178-1 by the first assessment (FIGS. 1B and 1C).

Conclusions—The P. obducens inoculum was shown to be viable following both inoculations with moderate to high levels of infection on control plants after the first inoculation and low to moderate levels of infection following the second inoculation. Under the conditions used in these tests no infection was observed on plants from test line 12-71-1 following inoculation with a metalaxyl-M resistant strain of P. obducens. Plants from test line 15-178-1 became infected following inoculation with the metalaxyl-M resistant strain of P. obducens, but it took longer to show symptoms than the control variety.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

Griffiths, A. & Matatiken, D. 2013. Impatiens gordonii. The IUCN Red List of Threatened Species 2013: e.T203725A2770808. dx.doi.org/10.2305/IUCN.UK.2013-1.RLTS.T203725A2770808.en. Downloaded on 16 November 2016.

Merlin, C. M., & Grant, W. F., “Hybridization studies in the genus Impatiens. Canadian Journal of Botany, 1986, Vol. 64, No. 5: pp. 1069-1074.

1. An Impatiens plant which exhibits tolerance to Plasmopara obducens infection wherein the plant is obtained by introgression of the tolerance trait from a plant grown from the seed deposited at NCIMB under the accession no. 42708, said plant having been selected for a Plasmopara obducens tolerant trait.

2. The Impatiens plant according to claim 1, wherein the plant exhibits infection less that 80% of the time after inoculation with Plasmopara obducens.

3. The Impatiens plant according to claim 1, wherein the plant exhibits complete tolerance to Plasmopara obducens infection.

4. The Impatiens plant according to claim 1, further defined as horticulturally elite.

5. The Impatiens plant according to claim 1, wherein the plant comprises a singe flower phenotype.

6. The Impatiens plant according to claim 1, wherein the plant comprises a double flower or semi-double phenotype.

7. A progeny plant of an Impatiens plant in accordance with claim 1, wherein said progeny plant exhibits tolerance to Plasmopara obducens infection.

8. A tissue culture of regenerable cells of the Impatiens plant of claim 1.

9. The tissue culture of claim 8, wherein the regenerable cells are from embryos, meristematic cells, pollen, leaves, petals, roots, root tips, anther, pistil, seed or stem.

10. A plant part of the Impatiens plant of claim 1.

11. The plant part of claim 10, wherein the part is a seed, a stalk, a petal, a bud, a leaf or a root.

12. The plant part of claim 10, wherein said part can be regenerated into a plant which exhibits tolerance to Plasmopara obducens infection.

13. A method of producing an Impatiens plant comprising crossing a plant of claim 1 with a second Impatiens plant and selecting a progeny plant which exhibits tolerance to Plasmopara obducens infection.

14. A method for obtaining an Impatiens plant according to claim 1 comprising the step of introgression of the tolerance trait from a plant grown from the seed deposited at NCIMB under the accession no. 42708.

15. An Impatiens seed wherein, upon growth, the seed produces a plant which exhibits a tolerance to Plasmopara obducens infection, said tolerance trait having been introgressed from a plant grown from the seed deposited at NCIMB under the accession no. 42708.
